(19) United States
(12) Patent Application Publication
(10) Pub. No.: US 2025/0017537 A1
(43) Pub. Date: Jan. 16, 2025

(54) PHYSICIAN NOTIFICATION MANAGEMENT

(71) Applicant: Cardiac Pacemakers, Inc., St Paul, MN (US)

(72) Inventors: Steven Esboldt, Oakdale, MN (US); Michael Thomas Edward McRoberts, Mazeppa, MN (US); Michael Rahn, Rochester, MN (US); Kelsey Morgan Johns, Millbury, MA (US); Peter O'Dea, St Paul, MN (US)

(21) Appl. No.: 18/767,723

(22) Filed: Jul. 9, 2024

Related U.S. Application Data
(60) Provisional application No. 63/526,017, filed on Jul. 11, 2023.

(51) Int. Cl. A61B 5/00; A61B 5/318; G16H 10/60; G16H 15/00

(52) U.S. Cl. CPC A61B 5/74; A61B 5/0006; A61B 5/0022; A61B 5/318; G16H 10/60; G16H 15/00

(57) ABSTRACT

Systems, methods, and devices involve approaches for limiting the number of detected cardiac events for which a physician is alerted. Approaches involve detecting cardiac events based on electrocardiogram (ECG) data and associating the cardiac events with a criticality. ECG data associated with a subset of the cardiac events is transmitted to a remote computing system based, at least in part, on the criticality. An electronic notification is transmitted to a patient care group regarding at least one of the cardiac events in the subset. Approaches further include determining whether an acknowledgement of the electronic notification has been received by the server from the patient care group within a set period of time.

### CROSS REFERENCE TO RELATED APPLICATION

This application claims priority to Provisional Application No. 63/526,017, filed Jul. 11, 2023, which is herein incorporated by reference in its entirety.

### TECHNICAL FIELD

The present disclosure relates to devices, methods, and systems for monitoring, identifying, classifying, and providing secure notifications and access to cardiac event information.

### BACKGROUND

Monitoring devices for collecting biometric data are becoming increasingly common in diagnosing and treating medical conditions in patients. For example, mobile devices can be used to monitor cardiac data in a patient. This cardiac monitoring can empower physicians with valuable information regarding the occurrence and regularity of a variety of heart conditions and irregularities in patients. Cardiac monitoring can be used, for example, to identify abnormal cardiac rhythms, so that critical alerts can be provided to patients, physicians, or other care providers and so that patients can be treated as needed.

### SUMMARY

In Example 1, a method includes detecting, by a server, cardiac events based on electrocardiogram (ECG) data and associating, using the server, the cardiac events with a criticality. The method further includes transmitting the ECG data associated with a subset of the cardiac events to a remote computing system based, at least in part, on the criticality. An electronic notification is transmitted to a patient care group regarding at least one of the cardiac events in the subset. The method further includes determining whether an acknowledgement of the electronic notification has been received by the server from the patient care group within a set period of time.

In Example 2, the method of Example 1, wherein the electronic notification does not include personal health information of a patient associated with the cardiac events.

In Example 3, the method of Example 2, wherein the personal health information can be accessed by one or more individuals in the patient care group via a mobile application.

In Example 4, the method of any of Examples 1-3, further including: after determining that the acknowledgement has not been received, transmitting electronic instructions to the remote computer to contact the patient care group by telephone.

In Example 5, the method of any of Examples 1-3, further including: after determining that the acknowledgement has been received, determining that the electronic notification has been successful.

In Example 6, the method of any of Examples 1-5, wherein the determining whether the acknowledgement has been received includes determining whether a mobile application user in the patient care group has: opened the mobile application, viewed the at least one of the cardiac events in the mobile application, or viewed a report associated with the at least one of the cardiac events in the mobile application.

In Example 7, the method of any of Examples 1-6, wherein the server operates a neural network to process the ECG data for the detecting the cardiac events.

In Example 8, the method of any of Examples 1-7, wherein the patient care group comprises a team of individuals within a practice.

In Example 9, the method of Example 8, wherein one or more of the individuals are associated with the patient care group by subscribing to the patient care group.

In Example 10, the method of Example 8, wherein one or more of the individuals are associated with the patient care group by delegation from a physician within the patient care group.

In Example 11, the method of Example 8, wherein one or more of the individuals are associated with the patient care group by forwarding the electronic notification by a physician within the patient care group.

In Example 12, the method of any of Examples 1-11, further including receiving a confirmation from the remote computer regarding at least one of the cardiac events in the subset of the cardiac events, wherein the transmitting the electronic notification occurs only after the confirmation is received.

In Example 13, a computer program product comprising instructions to cause one or more processors to carry out the steps of the method of Examples 1-12.

In Example 14, a computer-readable medium having stored thereon the computer program product of Example 13.

In Example 15, a server comprising the computer-readable medium of Example 14.

In Example 16, a system includes a server with one or more processors and with one or more computer-readable storage mediums storing instructions. When executed by the one or more processors, the instructions cause the server to perform an operation. The operation includes detecting cardiac events based on ECG data; associating the cardiac events with a criticality; transmitting the ECG data associated with a subset of the cardiac events to a remote computing system based, at least in part, on the criticality; receiving a confirmation from the remote computer regarding at least one of the cardiac events in the subset of the cardiac events; transmitting an electronic notification to a patient care group regarding the confirmation; and determining whether an acknowledgement of the electronic notification has been received from the patient care group within a set period of time.

In Example 17, the system of Example 16, wherein the electronic notification does not include personal health information of a patient associated with the cardiac events.

In Example 18, the system of Example 17, further including mobile devices associated with the individuals. Each mobile device includes a mobile application stored to memory, and the personal health information can be accessed only via the mobile application.

In Example 19, the system of Example 18, wherein the mobile application is programmed to provide access to a strip of ECG data associated with the at least one of the cardiac events in the subset of the cardiac events.

In Example 20, the system of Example 18, wherein the mobile devices and the server each include application program interfaces for enabling secure communication between the server and the mobile devices.

In Example 21, the system of Example 16, wherein the operation further includes: after determining that the acknowledgement has not been received, transmitting electronic instructions to the remote computer to contact the patient care group by telephone.

In Example 22, the system of Example 16, wherein the determining whether the acknowledgement has been received comprises determining whether a user in the patient care group has: opened a mobile application, viewed the at least one of the cardiac events in the mobile application, or viewed a report associated with the at least one of the cardiac events in the mobile application.

In Example 23, the system of Example 16, wherein the detecting the cardiac events and the associating the cardiac events with the criticality includes processing the ECG data with a neural network.

In Example 24, the system of Example 16, wherein the patient care group comprises a team of individuals within a practice, wherein any individual in the patient care group can initiate the acknowledgement.

In Example 25, a non-transitory computer-readable storage medium stores instructions, which, when executed by one or more processors, perform an operation. The operation includes detecting cardiac events based on ECG data; associating the cardiac events with a criticality; determining that the criticality breaches a threshold; transmitting an electronic notification to a patient care group regarding at least one of the cardiac events in the subset based on the breached threshold; and determining whether an acknowledgement of the electronic notification has been received from the patient care group within a set period of time.

In Example 26, the computer-readable storage medium of Example 25, wherein the electronic notification does not include personal health information of a patient associated with the cardiac events.

In Example 27, the computer-readable storage medium of Example 26, wherein the personal health information can be accessed by one or more individuals in the patient care group via a mobile application.

In Example 28, the computer-readable storage medium of Example 25, wherein the detecting the cardiac events and the associating the cardiac events with the criticality includes processing the ECG data with a neural network.

In Example 29, the computer-readable storage medium of Example 25, wherein the determining whether the acknowledgement has been received comprises determining whether a user in the patient care group has: opened a mobile application, viewed the at least one of the cardiac events in the mobile application, or viewed a report associated with the at least one of the cardiac events in the mobile application.

In Example 30, the computer-readable storage medium of Example 25, wherein the patient care group comprises a team of individuals within a practice.

In Example 31, the computer-readable storage medium of Example 30, wherein one or more of the individuals are associated with the patient care group by subscribing to the patient care group.

In Example 32, the computer-readable storage medium of Example 30, wherein one or more of the individuals are associated with the patient care group by delegation from a physician within the patient care group.

In Example 33, the computer-readable storage medium of Example 30, wherein one or more of the individuals are associated with the patient care group by forwarding the electronic notification by a physician within the patient care group.

In Example 34, the computer-readable storage medium of Example 30, wherein any individual in the patient care group can initiate the acknowledgement.

In Example 35, a method includes detecting, by a server, cardiac events based on ECG data and associating, using the server, the cardiac events with a criticality; transmitting the ECG data associated with a subset of the cardiac events to a remote computing system based, at least in part, on the criticality; transmitting an electronic notification to a patient care group regarding at least one of the cardiac events in the subset; and determining whether an acknowledgement of the electronic notification has been received by the server from the patient care group within a set period of time.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 shows a cardiac monitoring system, in accordance with certain instances of the present disclosure.

FIG. 2 shows a server, remote computer, and user interface, in accordance with certain instances of the present disclosure.

FIG. 3 shows an example portion of a user interface, in accordance with certain instances of the present disclosure.

FIG. 4 shows an example strip of ECG data and a summary of cardiac event data, in accordance with certain instances of the present disclosure.

FIG. 5 shows a flow diagram depicting an illustrative method of providing secure notifications and access to cardiac event information, in accordance with instances of the disclosure.

FIG. 6 shows a flow diagram of electronic notifications and acknowledgements, in accordance with certain instances of the present disclosure.

FIG. 7 is a block diagram depicting an illustrative computing device, in accordance with instances of the disclosure.

While the disclosed subject matter is amenable to various modifications and alternative forms, specific instances have been shown by way of example in the drawings and are described in detail below. The intention, however, is not to limit the disclosure to the particular instances described. On the contrary, the disclosure is intended to cover all modifications, equivalents, and alternatives falling within the scope of the disclosure as defined by the appended claims.

### DETAILED DESCRIPTION

Electrocardiogram (ECG) data of a patient can be used to identify whether the patient has experienced a cardiac event. To collect ECG data, one or more monitoring devices (e.g., sensors, mobile phones) can be coupled to the patient such that the monitoring devices sense and record the ECG data. The ECG data can be processed and analyzed by a computing device (e.g., a server) to detect whether the patient experienced a cardiac event. These detected cardiac events may ultimately be assessed by a physician to determine treatment for the patient. However, the number and frequency of detected cardiac events can be overwhelming to process and can bury the more critical cardiac events that may require treatment.

Instances of the present disclosure feature approaches for limiting the number of detected cardiac events for which a physician is alerted. Further, instances of the present disclosure features approaches for securely notifying and confirming receipt of the more critical cardiac events.

### Monitoring System

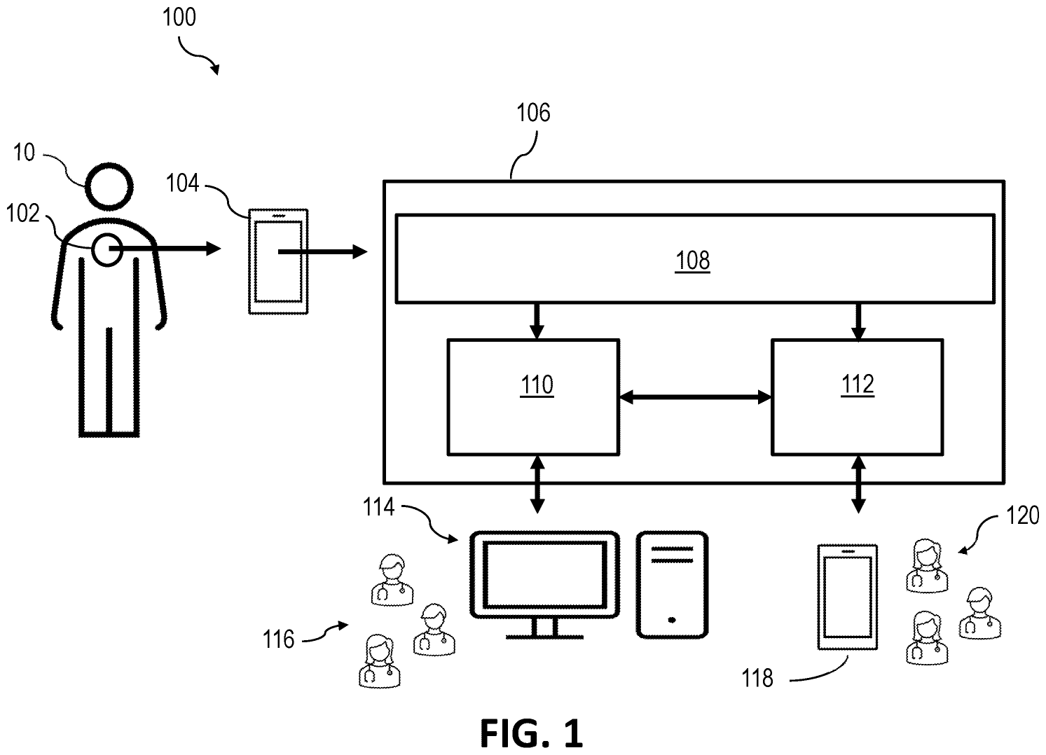

FIG. 1 illustrates a patient 10 and an example system 100 for monitoring patients' cardiac activity. The system 100 includes a monitor 102 attached to the patient 10 to detect cardiac activity of the patient 10. The monitor 102 may produce electric signals that represent the cardiac activity in the patient 10. For example, the monitor 102 may detect the patient's heart beating (e.g., using infrared sensors, electrodes) and convert the detected heartbeat into electric signals representing ECG data. The monitor 102 communicates the ECG data to a mobile device 104 (e.g., a mobile phone, a tablet).

The mobile device 104 may include a program (e.g., an application) that receives, processes, and analyzes the ECG data. For example, the program may analyze the ECG data and detect or flag cardiac events (e.g., periods of irregular cardiac activity) contained within the ECG data. As noted above, because ECG data may be getting continuously generated, the amount of ECG data can be overwhelming to store and process locally on the mobile device 104. As such, the mobile device 104 can periodically transmit chunks of the ECG data to another device or system, which can process, append together, and archive the chunks of the ECG data. In certain instances, the mobile device 104 is programmed to transmit short chunks of ECG data (e.g., 5- to 10-minute strips) along with metadata (e.g., time, duration, detected/flagged cardiac events) associated with the chunks of ECG data. In certain instances, the monitor 102 may be programmed to transmit the ECG data directly to the other device or system without utilizing the mobile device 104. Also, in certain instances, the monitor 102 and/or the mobile device 104 includes a button or touch-screen icon that allows the patient 10 to initiate an event. Such an indication can be recorded and communicated to the other device or system.

### Cardiac Event Server

In the example shown in FIG. 1, the mobile device 104 transmits the ECG data (and associated metadata, if any) to a cardiac event server 106 (hereinafter “the server 106” for brevity). The server 106 includes multiple platforms, layers, or modules that work together to process and analyze the ECG data such that cardiac events can be detected, filtered, prioritized, and ultimately reported to a patient's physician for analysis and treatment. In the example of FIG. 1, the server 106 includes one or more machine learning models 108 (e.g., deep neural networks), a cardiac event router platform 110, and a notification platform 112. As described in more detail below, the server 106 can provide access to ECG data, metadata, and summary information (e.g., reports) at a remote computer 114 for review by one or more users 116 (hereinafter the “user 116” for brevity) such as a team of technicians trained to process and analyze ECG data. Further, the server 106 can provide access to ECG data, metadata, and summary information to a remote/mobile device 118 (e.g., cell phone, tablet, laptop, computer, and the like) for review by one or more individuals of a patient care group 120 such as one or more physicians.

Although only one server 106 is shown in FIG. 1, the server 106 can include multiple separate physical servers, and the various platforms/modules/layers can be distributed among the multiple servers. Each of the platforms/modules/layers can represent separate programs, applications, and/or blocks of code where the output of one of the platforms/modules/layers is an input to another of the platforms/modules/layers. Each platform/module/layer can use application programming interfaces to communicate between or among the other platforms/modules/layers as well as systems and devices external to the server 106.

The server 106 applies the machine learning model 108 to the ECG data to classify cardiac activity of the patient 10. For example, the machine learning model 108 may compare the ECG data to labeled ECG data to determine which labeled ECG data the ECG data most closely resembles. The labeled ECG data may identify a particular cardiac event, including but not limited to ventricular tachycardia, bradycardia, atrial fibrillation, pause, normal sinus rhythm, or artifact/noise. In certain instances, the server 106 associates each detected cardiac event with a criticality (e.g., severity). The criticality can indicate whether a given cardiac event is considered a stable event, a critical event, or a serious event, among other potential labels for indicating criticality. In certain instances, the criticality of a given cardiac event is based on one or more characteristics such as whether a given patient is symptomatic or non-symptomatic and such as thresholds for heart rate and/or duration. These thresholds can be customized on a patient-by-patient basis or physician-by-physician basis. The determined criticality can be associated with the detected cardiac event as a metadata.

If the machine learning model 108 determines that the ECG data most closely resembles a labeled ECG data associated with a cardiac event, then the machine learning model 108 may determine that the patient 10 has experienced that cardiac event. Additionally, the machine learning model 108 may measure or determine certain characteristics of the cardiac activity of the patient 10 based on the ECG data. For example, the machine learning model 108 may determine a heart rate, a duration, or a beat count of the patient 10 during the cardiac event based on the ECG data. The machine learning model 108 may also determine a confidence level for each classification or identification of a cardiac event. The confidence level is an indication of certainty or uncertainty in the accuracy of the machine learning model's classification or identification. The server 106 stores the ECG data, the cardiac event, and associated metadata (e.g., information like heart rate, duration, beat count, criticality, etc.) in a database for storage. Subsequently, the server 106 may retrieve the cardiac event and associated information from the database.

### Pre-Notification Processing of ECG Data

The number and frequency of cardiac events detected by the server 106 can be overwhelming for a patient care group (e.g., one or more physicians) to process. Further, the amount of ECG data associated with all detected cardiac events can be overwhelming to transmit and store. As such, instead of sending the patient care group a notification and/or the ECG data for each detected event, the server 106 can facilitate efficient analysis and filtering of detected cardiac events that warrant notification and review by a patient care group.

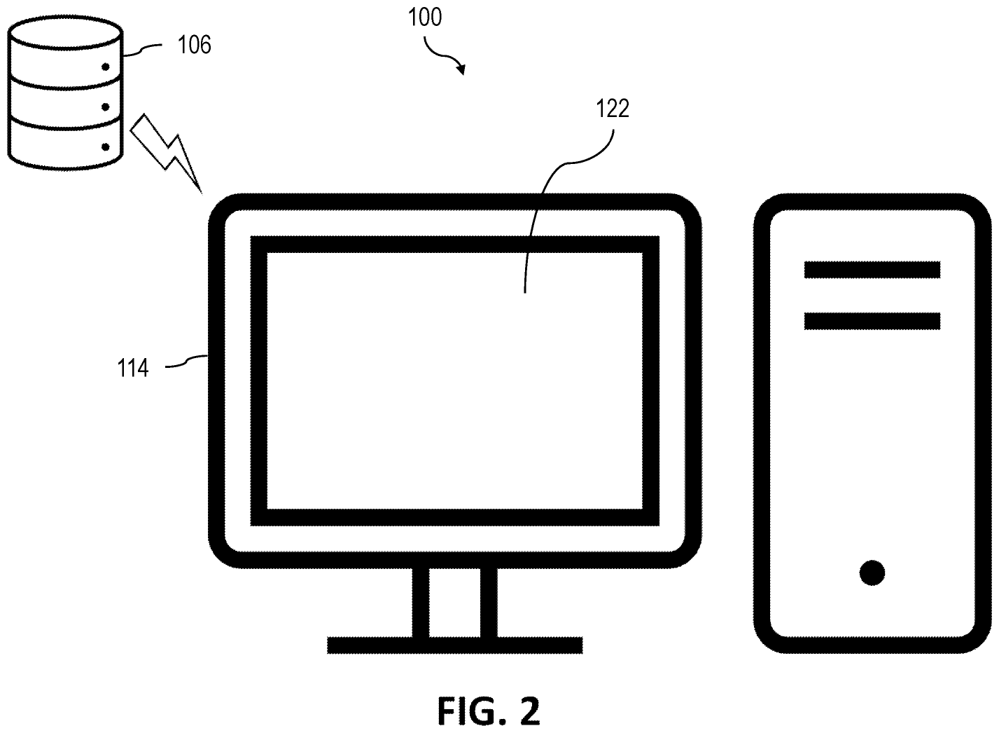

FIG. 2 shows the server 106 communicatively coupled (e.g., via a network) to the remote computer 114. In the example of FIG. 2, the remote computer 114 includes a monitor showing a user interface 122 for accessing, reviewing, analyzing, and modifying ECG data and metadata.

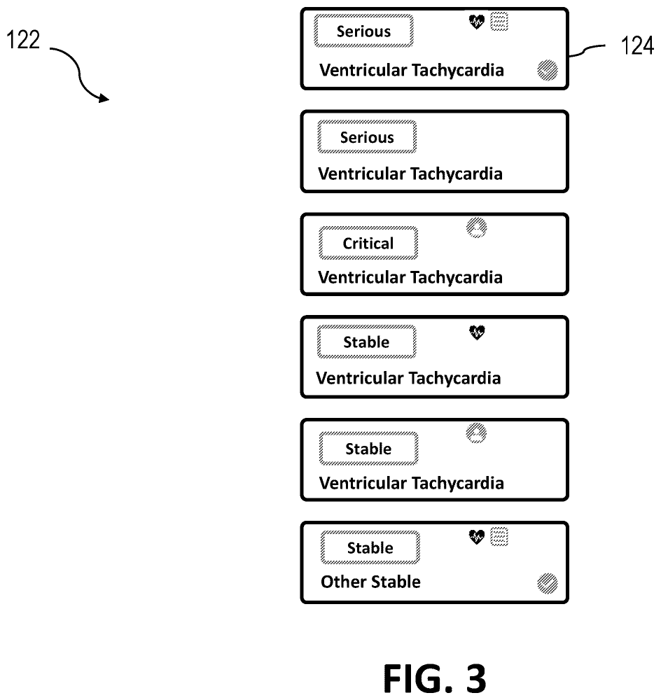

The server 106 (e.g., via the cardiac event router platform 110) can be programmed such that—when the server 106 (e.g., via the machine learning model 108) identifies a potential critical or serious cardiac event—the server 106 generates a new available session for selection by one of the users 116. The user 116 can select available sessions via the user interface 122. Once a session is selected, the server 106 can begin to transmit packages of data for use by the remote computer 114. In some instances, the remote computer 114 initiates the downloading of data by transmitting requests to the server 106. For example, as shown in FIG. 3, the user interface 122 can display a list of detected cardiac events 124 from which the user 116 can select. In certain instances, the list is initially sorted based on the criticality of the event (e.g., serious, critical, stable) as initially determined by the machine learning model 108. The list 124 can also indicate the type of rhythm (e.g., ventricular tachycardia, atrial fibrillation) initially assigned to the cardiac event by the machine learning model 108.

Once an event is selected by the user 116, the remote computer 114 can request the ECG data and associated metadata for that event from the server 106. To save computing resources and time, a relatively short strip of ECG data (e.g., 60 seconds) along with metadata and some ECG data surrounding the event can be downloaded-instead of downloading a large strip of ECG data (e.g., a 10-minute strip).

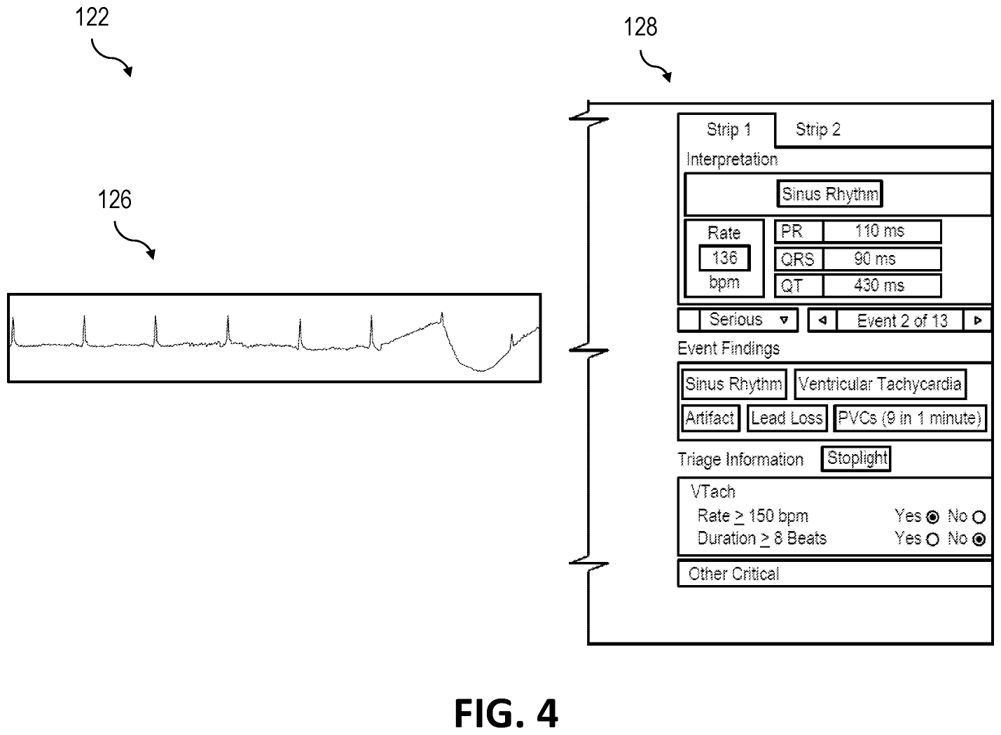

FIG. 4 shows an example of the type of data that can be displayed in the user interface 122 once the ECG data and associated metadata is downloaded at the remote computer 114. For example, a strip 126 of ECG data can be displayed as well as a summary 128 of metadata associated with the detected cardiac event.

The user interface 122 can allow the user 116 to scroll through the strip 126 of ECG data to view various time bands of the detected cardiac event. In the event the user 116 scrolls towards the boundaries of the downloaded strip 126 of ECG data, the remote computer 114 can transmit requests to the server 106 to download additional sections or strips of the ECG data so that the user's review is not interrupted or otherwise delayed by missing ECG data. Put another way, the ECG data can be downloaded on an as-needed basis in the background such that review is not delayed but also so that computing and storage resources are conserved by not downloading ECG data en masse.

Ultimately, the user 116 can determine whether one or more of the detected cardiac events warrants notification to the patient case group 120. In certain instances, the server 106 may support dozens, hundreds, or thousands of separate patient care groups. Each individual patient care group can set their own thresholds by patient, by physician, by clinic, etc., regarding what types or characteristics of cardiac events warrant notification. The user 116 can select such cardiac events. In response to the selection, the remote computer 114 can transmit a confirmation (e.g., a message/data) to the server 106, which initiates a secure notification process described below.

### Cardiac Event Notification

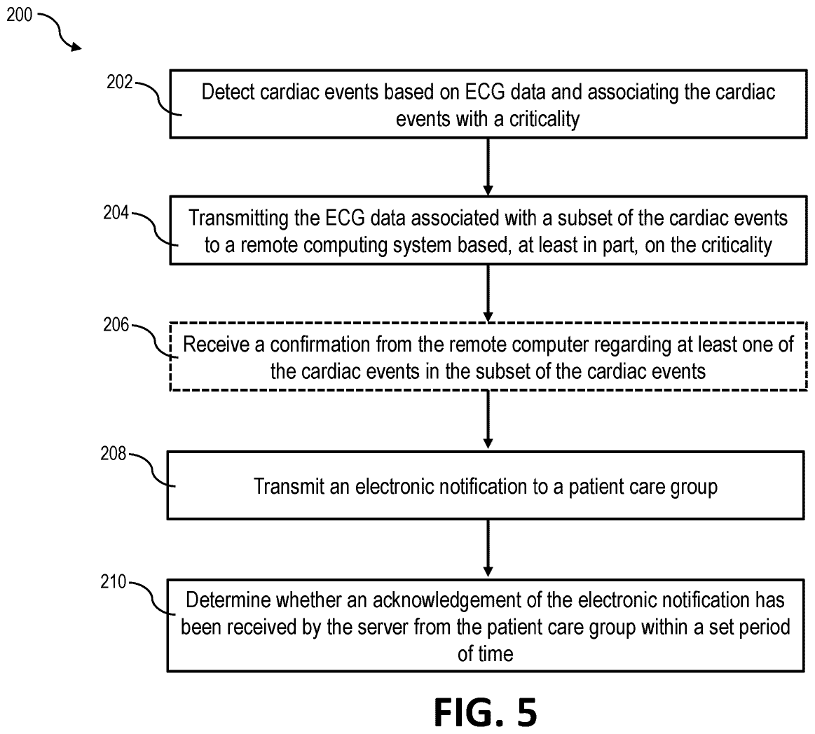

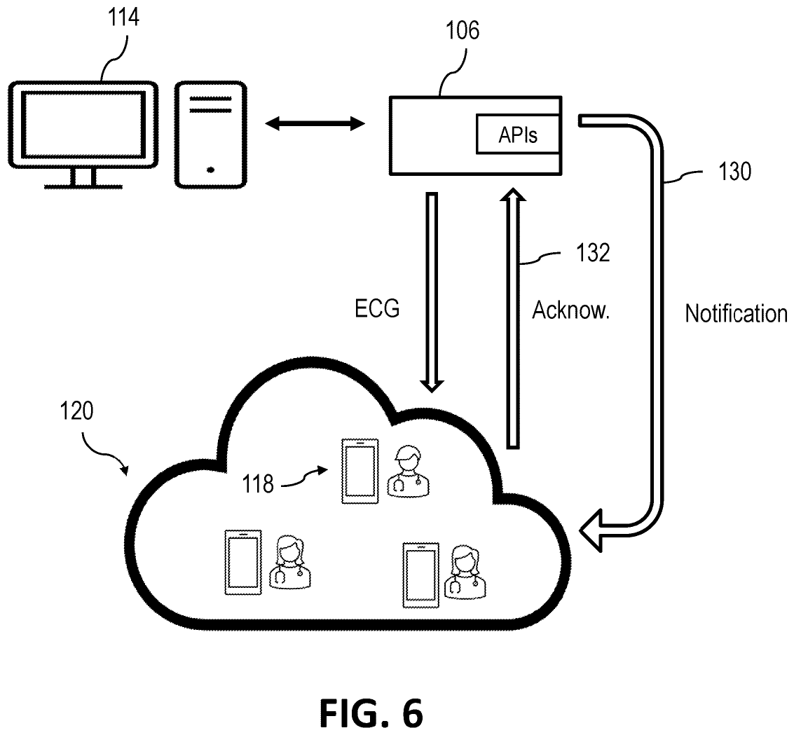

Examples of a cardiac event notification process are described below using the block diagram of FIG. 5 and the flow diagram of FIG. 6 as guides.

FIG. 5 shows a block diagram of a method 200 for a cardiac event analysis and notification process. As noted above, the server 106 (or another computing device) can first detect cardiac events based on ECG data and associate the cardiac events with a criticality (block 202 in FIG. 5). The server 106 can transmit the ECG data (and associated metadata) to the remote computer 114, where the data that is transferred is data associated with the cardiac events initially determined to be more serious or critical (block 204 in FIG. 5). As noted above, once at least one of the cardiac events is confirmed to warrant notifying a patient care group, the remote computer 114 can send a confirmation to the server 106 regarding the confirmed cardiac event (block 206 in FIG. 5). However, in some instances, an electronic notification can be initiated without receiving confirmation that such a notification is warranted. The server 106 can then initiate a secure electronic notification process by transmitting an electronic notification to the patient care group (block 208 in FIG. 5).

FIG. 6 shows a flow diagram of the cardiac event notification process. The server 106 can send an electronic notification 130 (e.g., a push notification) to the mobile device(s) 118 within a given patient care group 120. In certain instances, to receive electronic notifications, individuals in the patient care group 120 first download a mobile application to their mobile device 118. The mobile application can include an application programming interface (API) that enables the mobile device 118 to receive and utilize data structures of the electronic notifications generated by the server 106 and transmitted via the server's corresponding API. In other instances, the electronic notifications can be delivered by other means.

In certain instances, to protect privacy, the electronic notification 130 does not include personal information (e.g., personal health information) about the patient. Instead, the electronic notification 130 is formatted/structured such that the mobile devices 118 associated with the patient care group 120 generate a visual, physical, and/or audio alert (e.g., pop-up window, home screen icon, vibration, and/or noise and the like) on the mobile device 118. In response to the electronic notification 130, one or more individuals from the patient care group 120 can open the mobile application to view details about the notification. As such, although the electronic notification 130 does not include personal information, individuals from the patient care group 120 can access personal information and details about the detected cardiac event(s) via the mobile application. For example, the mobile application can provide access to ECG strips and associated metadata.

In certain situations, individuals in the patient care group 120 may fail to receive the electronic notification 130. For example, the mobile devices 118 associated with the patient care group 120 may temporarily not have access to a network (e.g., cellular, internet) such that the electronic notification 130 is not successfully delivered. As another example, the mobile devices 118 may be turned off and unable to receive the electronic notification 130. The cardiac event notification process, therefore, includes approaches to address situations where the electronic notification 130 is not received.

Referring back to FIG. 5, the method 200 includes determining whether an acknowledgement of the electronic notification 130 has been received by the server 106 from the patient care group 120 (block 210 in FIG. 5).

An acknowledgement 132 (as represented in FIG. 6) can be an electronic acknowledgement (e.g., message, data structure) that is generated by the mobile application and transmitted via an API to the server 106. The acknowledgement can be generated in response to several different actions. Examples include determining whether at least one of the mobile devices 118 (e.g., via the mobile application) in the patient care group 120 has successfully received the electronic notification 130 as evidenced by: the mobile device 118 generating an alert responsive to electronic notification 130, the mobile application being opened, the detected cardiac event being viewed via the mobile application, a report associated with the detected cardiac being viewed in the mobile application.

In certain instances, the server 106 is programmed to determine whether the acknowledgement 132 has been received from at least one mobile device 118 within a set period of time. If the acknowledgement 132 has been received, the cardiac event notification process can be completed. The server 106 can create an audit trail for each of the steps in the cardiac event notification process by recording data such as IP addresses, mobile phone IDs, user IDs, event IDs, time stamps, and the like so that notifications and acknowledgements are tracked.

If an acknowledgement has not been received (e.g., within a set period of time), the cardiac event notification process can initiate additional steps. As one example, one or more additional electronic notifications can be sent. As another example, if the first or subsequent electronic notifications are not acknowledged, the server 106 can create electronic instructions that instruct a user to contact the patient care group 120 directly (e.g., by telephone). This may include updating the user interface 122 (e.g., via an icon, message, text, and the like) at the remote computer 114 such that one or more of the users 116 can view the lack of receiving an acknowledgment and take appropriate action as instructed.

The patient care group 120 can be created using several different approaches. In certain instances, the initial individual in the patient care group 120 is the patient's primary physician who requested that the patient's cardiac activity be monitored. However, the primary physician may not always be available for notifications, and so additional individuals (e.g., other physicians within a practice/clinic) can be added to the patient care group 120 to receive electronic notifications. As one example, additional individuals can be added by subscribing to the patient care group 120 via the mobile phone application, which can verify that the additional individuals have permission to access a patient's personal information. As another example, additional individuals can be added by current members of the patient care group 120 delegating (e.g., via the mobile phone application) that the additional individuals are permitted to receive electronic notifications. As another example, current members of the patient care group 120 can set time periods (e.g., via the mobile phone application) where electronic notifications are automatically forwarded to other individuals. Any one of the individuals' mobile devices 118 in the patient care group 120 can initiate the acknowledgement 132.

The one or more servers 106 can service hundreds or thousands of individual patient care groups. As such, the various approaches described above can assist with processing and analyzing thousands and thousands of cardiac events and identifying which cardiac events ultimately warrant review by a patient care group.

### Computing Devices and Systems

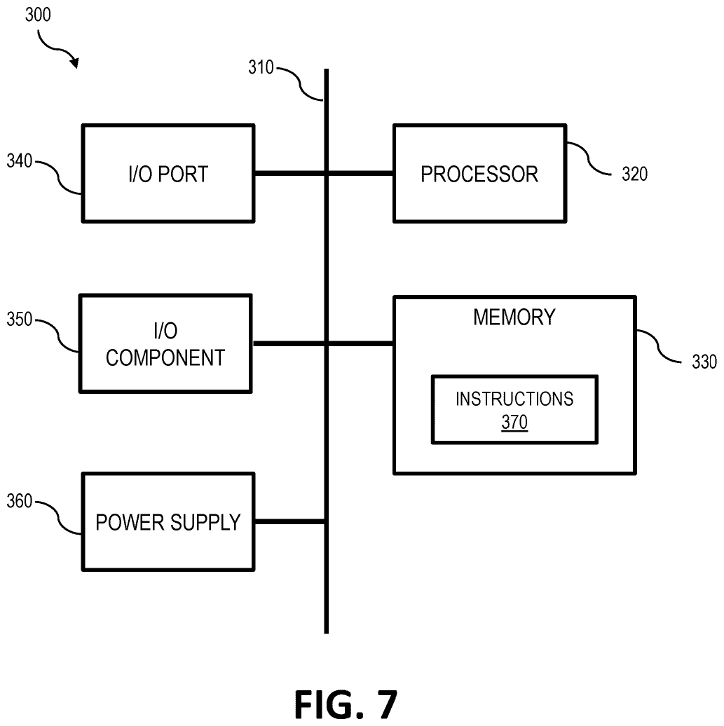

FIG. 7 is a block diagram depicting an illustrative computing device 300, in accordance with instances of the disclosure. The computing device 300 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 7 and described below. For example, the mobile devices, servers, and remote computers can each include their own set of components shown in FIG. 7 and described below.

In certain instances, the computing device 300 includes a bus 310 that, directly and/or indirectly, couples one or more of the following devices: a processor 320, a memory 330, an input/output (I/O) port 340, an I/O component 350, and a power supply 360. Any number of additional components, different components, and/or combinations of components may also be included in the computing device 300.

The bus 310 represents what may be one or more busses (such as, for example, an address bus, data bus, or combination thereof). Similarly, in instances, the computing device 300 may include a number of processors 320, a number of memory components 330, a number of I/O ports 340, a number of I/O components 350, and/or a number of power supplies 360. Additionally, any number of these components, or combinations thereof, may be distributed and/or duplicated across a number of computing devices.

In certain instances, the memory 330 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 330 stores computer-executable instructions 370 for causing the processor 320 to implement aspects of instances of components discussed herein and/or to perform aspects of instances of methods and procedures discussed herein. The memory 330 can comprise a non-transitory computer readable medium storing the computer-executable instructions 370.

The computer-executable instructions 370 may include, for example, computer code, machine-useable instructions, and the like such as, for example, program components capable of being executed by one or more processors 320 (e.g., microprocessors) associated with the computing device 300. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 370 may be configured to be executed by the processor 320 and, upon execution, to cause the processor 320 to perform certain processes. In certain instances, the processor 320, memory 330, and instructions 370 are part of a controller such as an application specific integrated circuit (ASIC), field-programmable gate array (FPGA), and/or the like. Such devices can be used to carry out the functions and steps described herein.

The I/O component 350 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a server including one or more processors and one or more computer-readable storage mediums storing instructions, which, when executed by the one or more processors, cause the server to perform an operation comprising:
detecting cardiac events based on electrocardiogram (ECG) data,
associating the cardiac events with a criticality,
transmitting the ECG data associated with a subset of the cardiac events to a remote computing system based, at least in part, on the criticality,
receiving a confirmation from the remote computer regarding at least one of the cardiac events in the subset of the cardiac events,
transmitting an electronic notification to a patient care group regarding the confirmation, and
determining whether an acknowledgement of the electronic notification has been received from the patient care group within a set period of time.

2. The system of claim 1, wherein the electronic notification does not include personal health information of a patient associated with the cardiac events.

3. The system of claim 2, further comprising:
mobile devices associated with the individuals, each mobile device including a mobile application stored to memory, wherein the personal health information can be accessed only via the mobile application.

4. The system of claim 3, wherein the mobile application is programmed to provide access to a strip of ECG data associated with the at least one of the cardiac events in the subset of the cardiac events.

5. The system of claim 3, wherein the mobile devices and the server each include application program interfaces for enabling secure communication between the server and the mobile devices.

6. The system of claim 1, wherein the operation further comprises:
after determining that the acknowledgement has not been received, transmitting electronic instructions to the remote computer to contact the patient care group by telephone.

7. The system of claim 1, wherein the determining whether the acknowledgement has been received comprises determining whether a user in the patient care group has: opened a mobile application, viewed the at least one of the cardiac events in the mobile application, or viewed a report associated with the at least one of the cardiac events in the mobile application.

8. The system of claim 1, wherein the detecting the cardiac events and the associating the cardiac events with the criticality includes processing the ECG data with a neural network.

9. The system of claim 1, wherein the patient care group comprises a team of individuals within a practice, wherein any individual in the patient care group can initiate the acknowledgement.

10. A non-transitory computer-readable storage medium storing instructions, which, when executed by one or more processors, perform an operation, the operation comprising:
detecting cardiac events based on electrocardiogram (ECG) data;
associating the cardiac events with a criticality;
determining that the criticality breaches a threshold;
transmitting an electronic notification to a patient care group regarding at least one of the cardiac events in the subset based on the breached threshold; and
determining whether an acknowledgement of the electronic notification has been received from the patient care group within a set period of time.

11. The computer-readable storage medium of claim 10, wherein the electronic notification does not include personal health information of a patient associated with the cardiac events.

12. The computer-readable storage medium of claim 11, wherein the personal health information can be accessed by one or more individuals in the patient care group via a mobile application.

13. The computer-readable storage medium of claim 10, wherein the detecting the cardiac events and the associating the cardiac events with the criticality includes processing the ECG data with a neural network.

14. The computer-readable storage medium of claim 10, wherein the determining whether the acknowledgement has been received comprises determining whether a user in the patient care group has: opened a mobile application, viewed the at least one of the cardiac events in the mobile application, or viewed a report associated with the at least one of the cardiac events in the mobile application.

15. The computer-readable storage medium of claim 10, wherein the patient care group comprises a team of individuals within a practice.

16. The computer-readable storage medium of claim 15, wherein one or more of the individuals are associated with the patient care group by subscribing to the patient care group.

17. The computer-readable storage medium of claim 15, wherein one or more of the individuals are associated with the patient care group by delegation from a physician within the patient care group.

18. The computer-readable storage medium of claim 15, wherein one or more of the individuals are associated with the patient care group by forwarding the electronic notification by a physician within the patient care group.

19. The computer-readable storage medium of claim 15, wherein any individual in the patient care group can initiate the acknowledgement.

20. A method comprising:
detecting, by a server, cardiac events based on electrocardiogram (ECG) data and associating, using the server, the cardiac events with a criticality;
transmitting the ECG data associated with a subset of the cardiac events to a remote computing system based, at least in part, on the criticality;
transmitting an electronic notification to a patient care group regarding at least one of the cardiac events in the subset; and
determining whether an acknowledgement of the electronic notification has been received by the server from the patient care group within a set period of time.
